(19) United States
(12) Patent Application Publication
(10) Pub. No.: US 2015/0011808 A1
(43) Pub. Date: Jan. 8, 2015

(54) Renewable High-Density, High-Octane Fuels

(71) Applicant: UNITED STATES OF AMERICA AS REPRESENTED BY THE SECRETARY OF THE NAVY, ARLINGTON, VA (US)

(72) Inventors: Benjamin G. Harvey, Ridgecrest, CA (US); Heather A. Meylemans, Ridgecrest, CA (US); Michael E. Wright, Ridgecrest, CA (US)

(21) Appl. No.: 14/171,855

(22) Filed: Feb. 4, 2014

Related U.S. Application Data
(60) Provisional application No. 61/763,203, filed on Feb. 11, 2013.

(51) Int. Cl. C07C 5/02; C10L 1/04

(52) U.S. Cl. CPC C07C 5/02; C10L 1/04
USPC 585/14; 585/254; 585/16

(57) ABSTRACT

A method/fuels for making high-density, high-octane fuels, the high-density, high-octane including, dimerizirig terpene monomer(s), crude mixture of terpene(s), and/or oxygenated terpenoid(s) with at least one heterogeneous dimerization acid catalyst at temperatures ranging from about 25° C. to about 160° C. to produce a mixture of residual/isomerized monomer(s) cymene(s), and terpene dimer(s), hydrogenating the mixture of residual/isomerized monomer(s), p-cymene(s), and terpene dimer(s) with at least one heterogenous catalyst(s) under a hydrogen atmosphere to produce a hydrogenated mixture of cymene(s), saturated cyclic molecules of terpene(s), other aromatic(s), and/or saturated terpene dimer(s), and isolating the hydrogenated mixture of cymene(s), saturated cyclic terpene(s), other aromatic(s), and saturated terpene dimer(s) by fractional distillation to yield a high boiling fraction composed of terpene dimers and mixture low boiling fraction composed of hydrogenated monomer(s) and cymenes.

### CROSS-REFERENCE TO RELATED APPLICATIONS

This is a non-provisional patent application, claiming the benefit of, parent application Ser. No. 61/763,203 filed on Feb. 11, 2013, whereby the entire disclosure of which is incorporated hereby reference.

### STATEMENT REGARDING FEDERALLY SPONSORED RESEARCH OR DEVELOPMENT

The invention described herein may be manufactured and used by or for the government of the United States of America for governmental purposes without the payment of any royalties thereon or therefor.

### FIELD OF THE INVENTION

The invention generally relates to processes for generating a high density gasoline from renewable terpenoids, and more specifically, fuels that have the potential to increase the range and/or time of flight of gasoline powered aerial vehicles.

### DETAILED DESCRIPTION OF EMBODIMENTS OF THE INVENTION

The invention generally relates to methods to generate renewable, high-density, high-octane fuels. These fuels can be generated from a variety of renewable terpenoid starting materials. Terpenoid-based fuels have the potential to meet or exceed the performance of current petroleum derived fuels.

Embodiments of the invention allow for generation of high octane fuels from the side-products generated in operations described in U.S. Pat. No. 8,227,651 and a non-provisional application submitted on Sep. 7, 2012. This important improvement allows for virtually 100% conversion of terpenoids into fuel products.

Previous work (U.S. Pat. No. 8,227,651) has shown that terpenoids can be converted to high density fuels by dimerization with heterogeneous acid catalysts. Some of the catalysts used for this reaction are also active for the dehydrogenation of the monomers to generate p-cymene. The residual low molecular weight products present after dimerization can be hydrogenated and combined to generate a high density, high-octane fuel. Utilization of the low molecular weight components of the process allows for a virtually quantitative conversion of terpenoids to renewable fuel products. The process can be modified to primarily produce gasoline-range fuels and a variety of starting materials including beta-pinene, alpha-pinene, limonene, camphene, fenchene, and crude turpentine can be utilized.

U.S. patent application Ser. No. 12/862,533 describes terpene based gasolines, but only as blendstocks with other high octane fuels. The current work describes methods to generate fully renewable, high-density, high-octane gasolines consisting of only hydrogenated terpenes and terpene derived molecules. In addition, this disclosure describes methods to synthesize the fuels either as side-products to other processes (high-density turbine fuels) or directly.

High density, high octane fuels are defined as having fuel densities of about 0.85 g/mL compared to conventional gasoline at 0.73 g/mL. Depending on the formulation, the high density, high octane fuels can have octane numbers greater than 90. Embodiments of the invention include formulations of about 50:50 (hydrogenated terpenes: aromatic terpene derivatives) and having an octane number of 91. Other embodiments of the invention include fuels with octane numbers between 85 and 100.

### Scheme 1.

1. A heterogeneous acid catalyst (acid clay, mesoporous zeolite, cation-exchange resin) is allowed to react with a terpene monomer (alpha-pinene, beta-pinene, limonene, camphene, fenchene, etc.), crude mixture of terpenes (turpentine), or various oxygenated terpenoids at a temperature in the range of about 25-170 degrees C. to generate a mixture of residual/isomerized terpenes, p-cymene and other cymenes, and terpene dimers.

2. The mixture is hydrogenated with a heterogeneous catalyst that may include Ni, Pd, Pt, Ru, Cu, Cr, Mo under a hydrogen atmosphere to generate a hydrogenated mixture of saturated terpenes, cymenes and other aromatics, and saturated terpene dimers.

3. The mixture of saturated monomers and cymene(s) are isolated by fractional distillation and can be directly used as gasoline. During distillation the “low boiling cut” is composed of cymenes and hydrogenated monomers and is suitable for gasoline. The “high boiling cut” is composed exclusively of terpene dimers and is useful as a diesel fuel additive or lubricant.

4. A high octane fuel can be formulated by increasing the amount of cymenes present in the distillate. This can be accomplished either by blending the fuel with additional cymenes or changing either the catalyst, starting terpene, or the conditions in step 1. These fuels can be blended with conventional fuels or other renewable fuels to meet performance requirements for specific operations.

### Alternate Method—Direct Synthesis of High Octane Terpenoid Gasoline:

1. A dehydrogenation catalyst (e.g. supported Pd or Pt, Lewis acids) is allowed to react with a terpene monomer or mixture of terpenoids at elevated temperature to generate a mixture of cymenes, isomerized terpenes, and menthenes.

2. The mixture is then placed under a hydrogen atmosphere and the non-aromatic hydrocarbons are then hydrogenated with a heterogeneous catalyst that ma, include Ni, Pd, Pt, Ru, Cu, Cr, Mo under a hydrogen atmosphere to generate a hydrogenated mixture of saturated terpenes, cymenes, other aromatics, and saturated terpene dimers

3. The octane number of the final fuel is controlled by choice of catalyst, terpene feedstock, and conditions employed in step 1. The octane number can be further controlled by adjustment of the concentration of cymenes in the fuel mixture. These fuels can be blended with conventional fuels or other renewable fuels to meet performance requirements for specific operations.

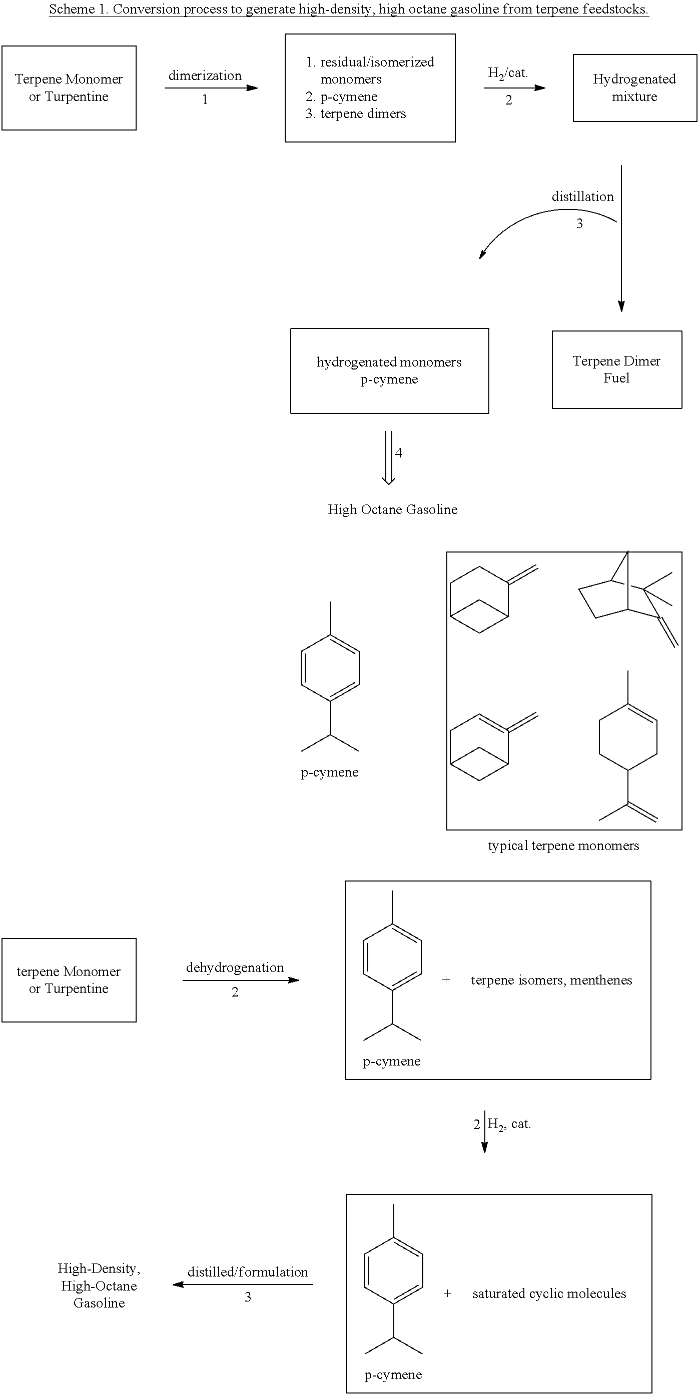

Embodiments of the invention generally relate to methods for making high-density, high-octane fuels including, dimerizing terpene monomer(s), crude mixtures of terpene(s), and/or oxygenated terpenoid(s) with at least one heterogeneous dimerization acid catalyst at temperatures ranging from about 25° C. to about 170° C. to produce a mixture of residual/isomerized monomer(s), cymene(s), and terpene dimer(s), hydrogenating the mixture of residual/isomerized monomer(s), cymene(s), and terpene dimer(s) with at least one heterogenous catalyst(s) under a hydrogen atmosphere to produce a hydrogenated mixture of cymene(s), saturated cyclic terpene(s), other aromatic(s), and/or saturated terpene dimer(s), and isolating the hydrogenated mixture of cymene(s), saturated cyclic terpene(s), other aromatic(s), and saturated terpene dimer(s) by fractional distillation to yield a high boiling fraction composed of terpene dimers and a low boiling fraction composed of hydrogenated monomer(s) and cymenes. The low boding fraction is collected from about 100 to 200° C. while the high boiling fraction is collected from about 280 to 350° C. at atmospheric pressure.

Other aspects of the invention include the fuel blends, high density, high octane fuels, and terpene dimer fuels produced by the methods herein. Still other aspects of the invention generally relate to methods for making high-density, high-octane fuels, including dehydrogenating and isomerizing terpene monomer(s), crude mixtures of terpene(s) or oxygenated terpenoids with at least one catalyst at elevated temperatures ranging from about 50° C. to about 170° C. to produce a mixture of menthenes, hydrogenated terpene(s), cymenes, other aromatics, and residual terpene isomer(s), hydrogenating the mixture of menthenes, hydrogenated terpene(s), cymenes, other aromatics, and residual terpene isomer(s) with at least one heterogenous catalyst(s) under a hydrogen atmosphere to produce a hydrogenated mixture of cymenes, other aromatics, saturated cyclic terpenes, and isolating the hydrogenated mixture of cymenes, other aromatics, and saturated cyclic terpene(s) by distillation to produce high-density, high octane fuels.

In embodiments, the terpene monomer(s) is selected from the group consisting of alpha-pinene, beta-pinene, limonene, camphene, fenchene, terpinenes, and any other combination thereof. In embodiments, the crude mixture of terpene(s) is selected from the group consisting of crude sulfate turpentine, gum turpentine, refined/purified turpentine distillates, and any other combination thereof. In embodiments, the terpene monomer(s) can be a terpenoid having ten carbons. In embodiments, the oxygenated terpenoid(s) is selected from the group consisting of cineoles, terpineols, cyclic terpene ethers and alcohols, and any other combination thereof. In embodiments, the heterogeneous dimerization acid catalyst(s) is selected from the group consisting of acid clay, zoolites, cation-exchange resins, sulfated zirconia, sulfated titania, polyphosphoric acid, supported mineral acids, supported Lewis acids, and any other combination thereof. In other embodiments, the heterogeneous catalyst(s) having transition metals selected from the group consisting of Pd, Pt, Ru, Ni, Cu, Mo, Cr, and any other combination thereof.

Embodiments further include increasing the cymene or aromatic concentration of the mixture of hydrogenated monomer(s) and cymene(s) based high-density, high-octane fuels to produce high octane fuels having an octane number ranging from about 85 to about 100. In other embodiments, the increasing cymene and aromatic content is achieved by blending said high-density, high-octane fuels with additional p-cymene (or other cymenes) or by altering the conditions (time/temperature) or the amount or type of heterogeneous catalyst. Embodiments further include blending the mixture of hydrogenated monomer(s) and cymene(s) having high-density and high-octane numbers with either renewable or petroleum-derived fuels to produce fuel blends. In embodiments, the dehydrogenation catalyst(s) having transition metals selected from the group consisting of Pd, Pt, Ni, Cu, Zn, and any other combination thereof.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

While the invention has been described, disclosed, illustrated and shown in various terms of certain embodiments or modifications which it has presumed in practice, the scope of the invention is not intended to be, nor should it be deemed to be, limited thereby and such other modifications or embodiments as may he suggested by the teachings herein are particularly reserved especially as they fall within the breadth and scope of the claims here appended.

What is claimed is:

1. A method for making a high-density, high-octane fuel, comprising:
dimerizing terpene monomer(s), crude mixture of terpene(s), and/or oxygenated terpenoid(s) with at least one heterogeneous dimerization acid catalyst at temperatures ranging from about 25° C. to about 170° C. to produce a mixture of residual/isomerized monomer(s), cymene(s), and terpene dimer(s);
hydrogenating said mixture of residual/isomerized monomer(s), cymene(s), and terpene dimer(s) with at least one catalyst under a hydrogen atmosphere to produce a hydrogenated mixture of cymene(s), saturated cyclic molecules of terpene(s), other aromatic(s), and/or saturated terpene dimer(s); and
isolating said hydrogenated mixture of cymene(s), saturated cyclic terpene(s), other aromatic(s), and saturated terpene dimer(s) by fractional distillation to yield a high boiling fraction composed of terpene dimers and boiling between about 280 and 350° C. under standard conditions, and a low boiling fraction composed of hydrogenated monomer(s) and cymenes and boiling between about 100 and 200° C.

2. The method according to claim 1, wherein said terpene monomer(s) is selected from the group consisting of alpha-pinene, beta-pinene, limonene, camphene, fenchene, terpinenes, and any other combination thereof.

3. The method according to claim 1, wherein said terpene monomer(s) is a terpenoid having ten carbons.

4. The method according to claim 1, wherein said crude mixture of terpene(s) is selected from the group consisting of crude sulfate turpentine, gum turpentine, refined/purified turpentine distillates, and any other combination thereof.

5. The method according to claim 1, wherein said oxygenated terpenoid(s) is selected from the group consisting of cineoles, terpineols, cyclic terpene ethers and alcohols, and any other combination thereof.

6. The method according to claim 1, wherein said heterogeneous dimerization acid catalyst(s) is elected from the group consisting of acid clay, zeolites, cation-exchange resins, sulfated zirconia, sulfated titania, polyphosphoric acid, supported mineral acids, supported Lewis acids, and any other combination thereof.

7. The method according to claim 1, wherein said heterogeneous catalyst(s) having transition metals selected from the group consisting of Pd, Pt, Ru, Ni, Cu, Cr, Mo under a hydrogen atmosphere to generate a hydrogenated mixture of saturated terpenes, emetics, other aromatics, and saturated terpene dimers, any other combination thereof.

8. The method according to claim 1, further comprising increasing said cymene or aromatic concentration of said mixture of hydrogenated monomer(s) and cymene(s) based high-density, high-octane fuels to produce high octane fuels having an octane ranging from about 85 to about 100.

9. The method according to claim 8, wherein said increasing cymene and aromatic content is achieved by blending said high-density, high-octane fuels with additional p-cymene or by altering the conditions (time/temperature) of the dimerization reaction or the amount or type of heterogeneous catalyst.

10. The method according to claim 1, further comprising blending said mixture of hydrogenated monomer(s) and cymene(s) having high-densities and high-octane numbers with either renewable or petroleum-derived fuels to produce fuel blends.

11. Fuel blends produced by the methods of claim 10.

12. High density, high octane fuels produced by the methods of claim 1.

13. Terpene dimer fuels produced by the methods of claim 1.

14. A method for making high-density, high-octane fuels, comprising:
dehydrogenating and isomerizing terpene monomer(s), crude mixtures of terpene(s) or oxygenated terpenoids with at least one dehydrogenation catalyst at elevated temperatures ranging from about 50° C. to about 170° C. to produce a mixture of menthenes, hydrogenated terpene(s), cymenes, other aromatics, and residual terpene isomer(s);
hydrogenating said mixture of menthenes, hydrogenated terpene(s), cymenes, other aromatics, and residual terpene isomer(s) with at least one heterogenous catalyst(s) under a hydrogen atmosphere to produce a hydrogenated mixture of cymenes, other aromatics, saturated cyclic terpenes; and
isolating said hydrogenated mixture of cymenes, other aromatics, saturated cyclic terpene(s) by distillation to produce high-density, high octane fuels.

15. The method according to claim 14, wherein said terpene monomer(s) is selected from the group consisting of alpha-pinene, beta-pinene, limonene, camphere, fenchene, terpinenes, and any other combination thereof.

16. The method according to claim 14, wherein said terpene monomer(s) having ten carbons.

17. The method according to claim 14, wherein said crude mixture of terpene(s) is selected from the group consisting of crude sulfate turpentine, gum turpentine, and refined/purified turpentine distillates, and any other combination thereof.

18. The method according to claim 14, wherein said oxygenated terpenoid(s) is selected from the group consisting of cineoles, terpineols, cyclic terpene ethers and alcohols, and any other combination thereof.

19. The method according to claim 14, wherein said dehydrogenation catalyst(s) having transition metals selected from the group consisting of Pd, Pt, Ni, Cu, Zn, and any other combination thereof.

20. The method according to claim 14, wherein said heterogeneous hydrogenation catalyst(s) is selected from the group consisting of Pd, Pt, Ru, Ni, Cu, Cr, Mo under a hydrogen atmosphere to generate a hydrogenated mixture of saturated terpenes, cymenes, other aromatics, and saturated terpene dimers, any other combination thereof.

21. The method according to claim 14, further comprising increasing the amount of cymenes of said mixture of p-cymenes, aromatics, and saturated cyclic terpene(s) to produce high octane fuels having an octane ranging from about 85 to about 100.

22. The method according to claim 14, wherein said increasing p-cymene is by blending said high-density, high-octane fuels with additional cymene or other like cymenes.

23. The method according to claim 14, further comprising blending said hydrogenated mixture of cymenes, other aromatics, saturated cyclic terpene(s) with other renewable or petroleum based fuels to produce fuel blends.

24. Fuel blends produced by the methods of claim 22.

25. High density, high octane fuels produced by the methods of claim 21.
